(19) United States
(12) Patent Application Publication
(10) Pub. No.: US 2018/0333148 A1
(43) Pub. Date: Nov. 22, 2018

(54) SYSTEM AND METHOD FOR QUANTIFIABLE SHAVE BIOPSY

(71) Applicants: Indiana University Research and Technology Corporation, Indianapolis, IN (US); Rose-Hulman Institute of Technology, Terre Haute, IN (US)

(72) Inventors: Luke G. Gutwein, Franceville, IN (US); Sunil Tholpady, Carmel, IN (US); Barry Davignon, Terre Haute, IN (US)

(73) Assignees: Indiana University Research and Technology Corpora tion, Indianapolis, IN (US); Rose-Hulman Institute of Technology, Terre Haute, IN (US)

(21) Appl. No.: 15/767,139

(22) PCT Filed: Oct. 17, 2016
(86) PCT No.: PCT/US16/57367

§ 371 Date: Apr. 9, 2018

Related U.S. Application Data
(60) Provisional application No. 62/242,125, filed on Oct. 15, 2015.

(51) Int. Cl. A61B 10/02

(52) U.S. Cl. CPC A61B 10/0283; A61B 2010/0208

(57) ABSTRACT

Systems and methods for shave biopsy are disclosed for extracting tissue at quantifiable specimen depth at the time of tissue acquisition. In particular, a biopsy apparatus is provided that generally includes a suction mechanism and a shaving mechanism.

### PRIORITY CLAIM

This application claims priority to U.S. 62/242,125 filed on Oct. 15, 2015, the entire disclosure of which is hereby expressly incorporated by reference herein.

### FIELD OF DISCLOSURE

The present disclosure relates generally to biopsies, particularly to systems and methods for extracting tissue samples at quantifiable depths via shave biopsies.

### BACKGROUND OF DISCLOSURE

One of the most common human cancers is skin cancer. For example, some 2 million diagnoses of skin cancer were made in 2010 alone. Melanoma is the most lethal skin cancer and third most common skin cancer. Furthermore, melanoma incidences are rapidly rising with a 600% increase from 1950 to 2000. Prognosis of malignant melanoma is directly related to the depth of tumor invasion. Clinical management is dependent on and guided by an accurate depth of invasion as assessed by a biopsy.

Generally, a skin cancer diagnosis is made following results of a skin biopsy. The most common biopsy procedure is a shave biopsy procedure. The shave biopsy procedure typically involves sliding a razor blade back and forth by hand to remove a tissue sample from a patient. Advantageously, shave biopsies require few stitches when compared to alternative techniques, and sometimes none at all. Unfortunately, traditional shave techniques are hampered by an inability to ensure an accurate biopsy depth. For instance, when a skin lesion is biopsied using a traditional shave biopsy technique and is of insufficient depth, the pathological analysis will identify malignant melanoma with a positive deep margin. This translates to cancer left behind and an unknown depth of invasion. As a result, an additional biopsy procedure or a surgery may be needed because of the inadequacy of the initial shave biopsy. This increases healthcare expenditures and delays timely definitive therapy for the melanoma patient.

Simplistically, critical decision points in melanoma management involve a depth of invasion of 1 millimeter or greater. A greater than 1 millimeter depth of invasion implies the need for a larger area of skin resection, as well as lymph node basin sampling. As a result, current medical/surgical dogma dictates that the clinician use a punch biopsy to study any suspicious lesion. This is because the punch biopsy provides full depth information for future intervention. Unfortunately, the relative invasiveness and requisite stitches cause many patients and clinicians to favor the traditional shave biopsy, despite the above-described drawbacks of the shave biopsy.

Therefore, it would be advantageous to have a system and method for performing biopsies that is capable of securing tissue samples at a quantifiable depth that can provide necessary clinical and pathological information, while reducing the invasiveness and/or cosmetic impact on the patient.

### SUMMARY OF THE DISCLOSURE

In various embodiments, a biopsy apparatus for extracting a quantifiable depth of tissue from skin of a patient may be provided that includes a suction mechanism and a shaving mechanism, whereby the depth of the biopsy is quantified. The suction mechanism being coupled to a vacuum source, and comprising a conduit having a proximal end and a distal end, a channel extending from the distal end of the conduit, and a screen in fluid communication with the channel and the conduit, and the shaving mechanism including a blade.

In one embodiment, the shaving mechanism further comprises an elongated member coupled to the blade.

In another embodiment, the screen is positioned between the distal end of the conduit and the channel.

In a further embodiment, the screen is laterally offset from the conduit.

In another embodiment, the biopsy apparatus further includes a connecting mechanism comprising at least one link coupling the shaving mechanism and the suction mechanism.

In certain embodiments, the channel is created by a perforated plate.

In a further embodiment, the biopsy apparatus further includes an actuating mechanism coupled to the shaving mechanism, whereby the actuating mechanism engages the blade to rotate and move the blade across the skin to remove a portion of skin suctioned by the suction mechanism.

In another embodiment, the actuating mechanism is one of a lever and a motor.

In a further embodiment, the motor includes a power source and a power switch.

In another embodiment, the power source is a battery.

In a certain embodiments, the power switch is positioned about an exterior surface of the elongated member.

In another embodiment, the shaving mechanism further includes a support plate coupled to the blade.

In a certain embodiments, the blade is round.

In another embodiment, the screen is formed within a plate.

According to another exemplary embodiment of the present disclosure, a method for extracting a quantifiable depth of tissue at harvest comprises providing a biopsy apparatus, the biopsy apparatus comprising a suction mechanism coupled to a vacuum source and a shaving mechanism, the suction mechanism comprising a conduit, a channel and a screen, wherein the conduit has a proximal end and a distal end, and the screen is in fluid communication with the channel and the conduit, and the shaving mechanism comprising a blade, positioning the suction mechanism of the biopsy apparatus adjacent to skin of a patient, actuating the vacuum source, whereby suction is applied to the skin of the patient through the conduit and the channel creating a suctioned portion of skin within the channel, and actuating the shaving mechanism, whereby the blade passes over the skin tangential to the suctioned portion of skin and under the suction mechanism thereby removing the suctioned portion of skin from the patient.

In one embodiment, the method further comprises pulling the suction mechanism away from the skin of the patient prior to actuating the shaving mechanism.

In another embodiment, the shaving mechanism is actuated by rotation of a lever coupled to the elongated member.

In certain embodiments, the shaving mechanism is actuated by initiating a motor coupled to the elongated member.

In another embodiment, the motor is initiated by toggling a power switch coupled to the elongated member.

In certain embodiments, the screen is positioned between the distal end of the conduit and the channel, and a depth of the suctioned portion of skin is determined by the screen.

Additional features and advantages of the present disclosure will become apparent to those skilled in the art upon consideration of the following detailed description of the illustrative embodiment exemplifying the best mode of carrying out the disclosure as presently perceived.

### BRIEF DESCRIPTION OF THE DRAWINGS

The foregoing aspects and many of the intended advantages of this disclosure will become more readily appreciated as the same becomes better understood by reference to the following detailed description when taken in conjunction with the accompanying drawings.

FIG. 1 is a perspective view of an embodiment of a biopsy apparatus of the present disclosure in an open position;

FIG. 2 is a perspective view of another embodiment of a biopsy apparatus of the present disclosure in an open position;

FIG. 3 is a perspective view of another embodiment of a biopsy apparatus of the present disclosure in an open position;

FIG. 4 is a perspective view of another embodiment of a biopsy apparatus of the present disclosure in an open position;

FIG. 5 is another perspective view of the biopsy apparatus of FIG. 2;

FIG. 6 is another perspective view of the biopsy apparatus of FIG. 3;

FIG. 7 is another perspective view of the biopsy apparatus of FIG. 4;

FIG. 8a-c is a perspective view of the biopsy apparatus of FIG. 1 transitioning from an open position (FIG. 8a) to a midway position (FIG. 8b) to a closed position (FIG. 8c);

FIG. 9a-c is a perspective view of the biopsy apparatus of FIG. 2 transitioning from an open position (FIG. 9a) to a midway position (FIG. 9b) to a closed position (FIG. 9c);

FIG. 10a-c is a perspective view of the biopsy apparatus of FIG. 3 transitioning from an open position (FIG. 10a) to a midway position (FIG. 10b) to a closed position (FIG. 10c); and

FIG. 11a-c is a perspective view of the biopsy apparatus of FIG. 4 transitioning from an open position (FIG. 11a) to a midway position (FIG. 11b) to a closed position (FIG. 11c).

Corresponding reference characters indicate corresponding parts throughout the several views. Although the drawings represent embodiments of various features and components according to the present disclosure, the drawings are not necessarily to scale and certain features may be exaggerated in order to better illustrate and explain the present disclosure. The exemplification set out herein illustrates embodiments of the disclosure, and such exemplifications are not to be construed as limiting the scope of the disclosure in any manner.

### DETAILED DESCRIPTION

For the purposes of promoting an understanding of the principals of the disclosure, reference will now be made to the embodiments illustrated in the drawings, which are described below. The embodiments disclosed below are not intended to be exhaustive or limit the disclosure to the precise form disclosed in the following detailed description. Rather, the embodiments are chosen and described so that others skilled in the art may utilize their teachings. It will be understood that no limitation of the scope of the disclosure is thereby intended. The disclosure includes any alterations and further modifications in the illustrative apparatuses and described methods and further applications of the principles of the disclosure which would normally occur to one skilled in the art to which the disclosure relates.

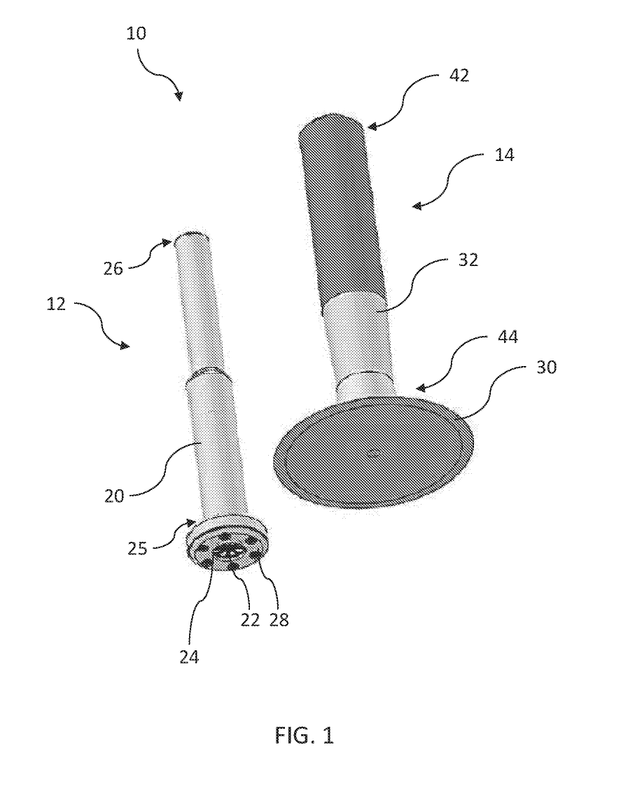

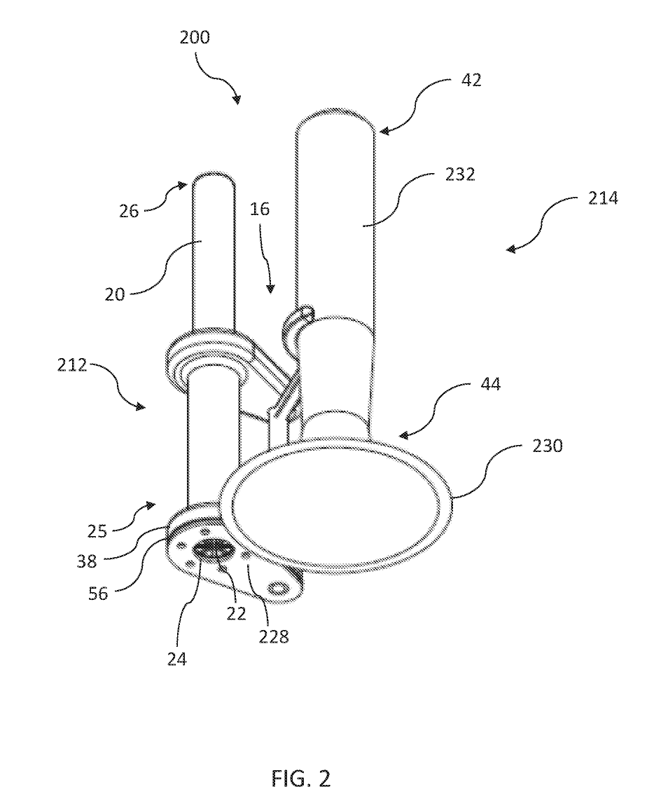

Referring to FIGS. 1-4, a biopsy apparatus 10, 200, 300, 400 for extracting a quantifiable depth of tissue generally includes a suction mechanism 12, 212, 312, 412 and a shaving mechanism 14, 214, 314, 414. Additionally, and referring to FIGS. 2-4, biopsy apparatus 200, 300, 400 may, in various embodiments, also include a connecting mechanism 16, 316, 416. In various embodiments, suction mechanism 12, 212, 312, 412 and/or connecting mechanism 216, 316, 416 may be constructed on a 3-D printer. Furthermore, suction mechanism 12, 212, 312, 412 and connecting mechanism 16, 316, 416 may be formed from a polypropylene material, or other suitable material(s).

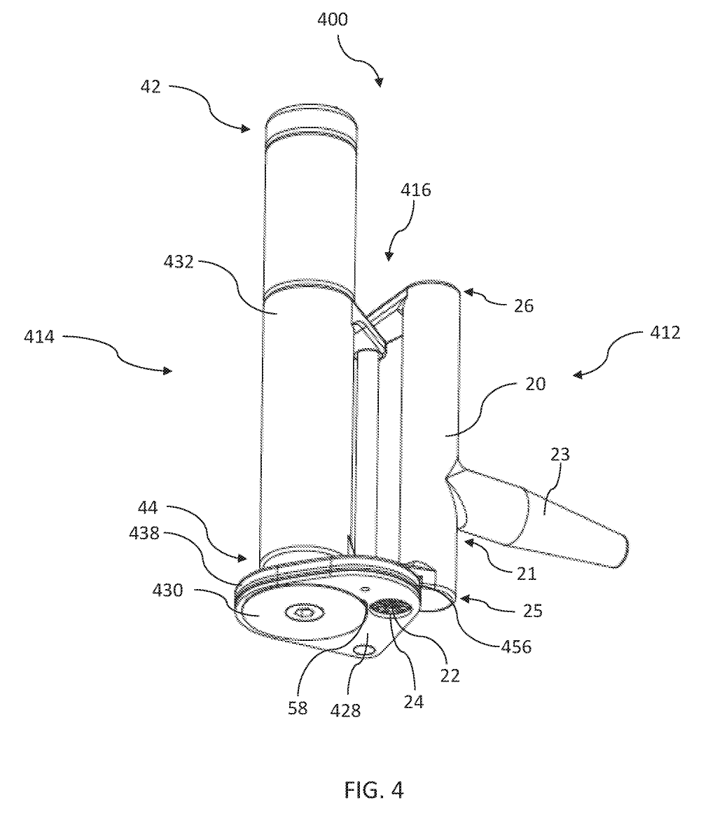

In more detail, and still referring to FIGS. 1-4, suction mechanism 12, 212, 312, 412 generally includes a conduit 20, a screen 22, and a channel 24, wherein conduit 20 has a distal end 25 and a proximal end 26. Generally, screen 22 may be situated between channel 24 and distal end 25 of conduit 20. Additionally, screen 22 may be attached or abutted to distal end 25 of conduit 20. In various embodiments, screen 22 may be laterally offset from conduit 20 such that a longitudinal axis of conduit 20 is parallel to a longitudinal axis of screen 22 and channel 24. Furthermore, in various embodiments, distal end 25 of conduit 20 may be of greater circumference than proximal end 26. In addition, in various embodiments, proximal end 26 of conduit 20 may be in communication with a tube or a hose (not shown). The tube or hose may be connected to a suction source (not shown), wherein the suction source provides a lifting force on the skin of the patient through conduit 20 such that the skin may be drawn into channel 24 until stopped by screen 22. In an exemplary embodiment, the suction source is a vacuum pump. In other various embodiments, suction mechanism 12 may include an opening 21 along a side surface of conduit 20 in communication with the tube or hose of the suction source (FIG. 4). Opening 21 may include a conduit 23 extending outward from conduit 20. When suction mechanism 12 includes opening 21 along the side of conduit 20, proximal end 26 of conduit 20 is sealed or closed off.

Furthermore, and still referring to FIGS. 1-4, channel 24 may be formed such that it allows for conduit 20 of suction mechanism 12, 212, 312, 412 to obtain a sealed vacuum with the skin of a patient when in use, wherein screen 22 may stop the skin from getting suctioned into conduit 20 too far resulting in the removal of excess skin. Additionally, channel 24 may be sufficiently elongated from screen 22 to accommodate the appropriate depth of tissue that needs to be suctioned and removed from a patient as is further described below. Generally, the length of channel 24 is engineered such that the depth of the tissue when harvested is at least 1.1 millimeters from the stratum corneum (surface of skin). Additionally, the diameter of the tissue harvested may be determined by the diameter of channel 24, wherein the diameter of the channel is generally about 9.5 millimeters.

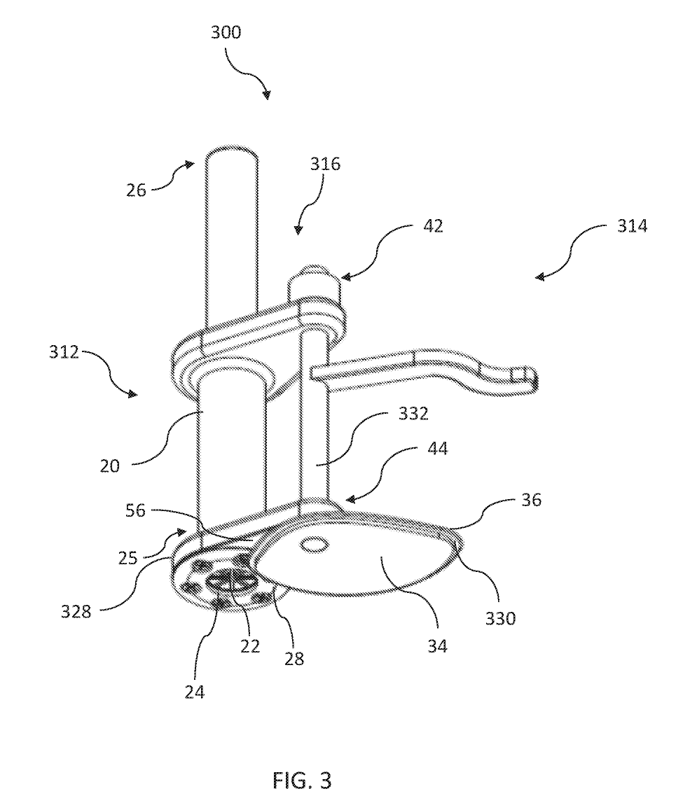

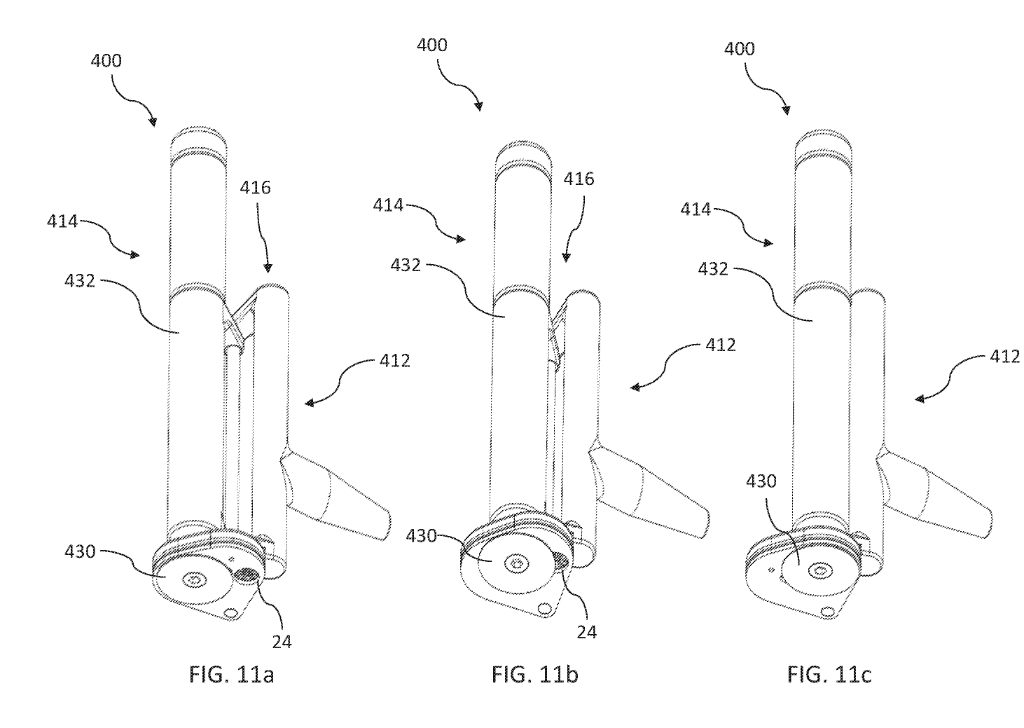

Furthermore, channel 24 may be formed through a perforated plate 28, 228, 328, 428 or an extending member (not shown). In various embodiments, perforated plate 28 may be rounded and cover a substantial portion of distal end 25 of conduit 20 (FIG. 1), or perforated plate 328 may cover only a portion of a bottom of a link 338 of connecting mechanism 316 situated near distal end 25 of conduit 20 (FIG. 3). In an example embodiment, perforated plate 28, 328 may be circular. Additionally, in other various embodiments, perforated plate 228, 428 may be congruent with the bottom of link 38, 438 (FIGS. 2 and 4). Beyond this, screen 22 may also be integral with perforated plate 28 wherein screen 22 may be coupled at a proximal end of channel 24 adjacent to conduit 20 while skin of a patient would abut a distal end of channel 24 when apparatus 10 is in use. Furthermore, as can be seen in FIGS. 2 and 4, screen 22 may, in various embodiments, be integrated into a second perforated plate 56, 456 such that second perforated plate 56, 456 is congruent with a bottom surface of link 38, 438 and situated between distal end 25 of conduit 20 and/or link 38/438 of connecting mechanism 16 and perforated plate 228, 428. Perforated plate 456 may further include an opening 58 (FIG. 4) configured to receive an arm extending from a motor (not shown) positioned within elongated member 432, which guides the arm and blade 430 from an open position (FIG. 11a) to a closed position (FIG. 11c).

With reference still to FIGS. 1-4, shaving mechanism 14, 214, 314, 414 generally includes a blade 30, 230, 330, 430 and an elongated member 32, 232, 332, 432. Generally, blade 30, 230, 330, 430 may be rounded. In various embodiments, blade 30, 230, 330, 430 may be circular or substantially oval. In certain embodiments, blade 30, 230, 330, 430 may be a custom fabricated scalpel blade. Furthermore, in various embodiments, blade 30, 230, 330, 430 may be a 28 mm blade or a 45 mm blade.

Furthermore, elongated member 32, 232, 332, 432 is generally coupled to blade 30, 230, 330, 430. Additionally, elongated member 32, 232, 332, 432 may be coupled to a top surface (not shown) or through an opening in blade 30, 230, 330, 430, wherein a bottom surface of blade 30 glides across the top of the skin of a patient when shaving mechanism 14, 214, 314, 414 is actuated. In various embodiments, blade 430 may be moveably coupled to elongated member 432 beneath perforated plate 428 such that no portion of blade 430 extends beyond outer edges of perforated plate 428. In addition, elongated member 32, 232, 332, 432 may be fixedly coupled to blade 30, 230, 330, 430 such that when elongated member 32, 232, 332, 432 is rotated so too is blade 30, 230, 330, 430. In addition, elongated member 32 may include a proximal end 42 and a distal end 44, wherein proximal end 42 may have a larger, smaller, or equal circumference when compared to distal end 44.

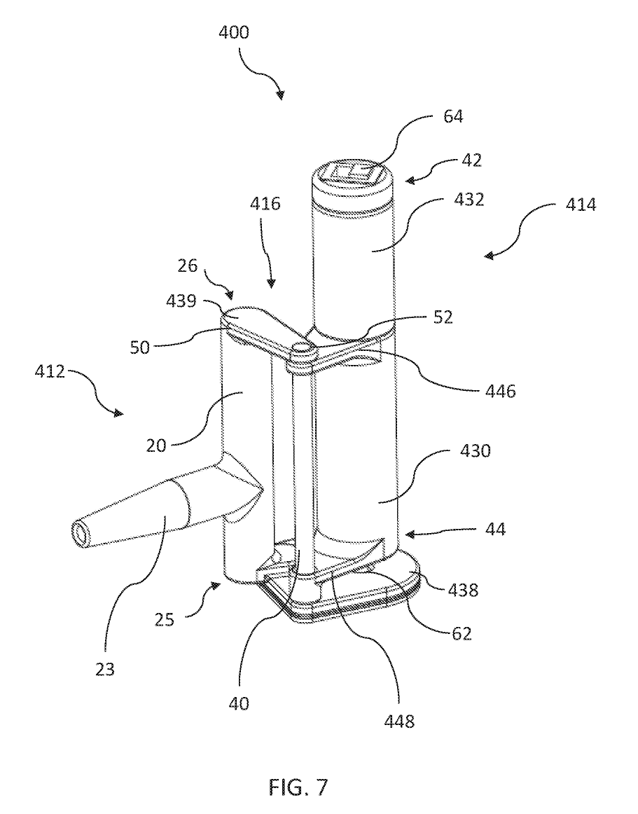

Further, in various embodiments, shaving mechanism 14 may be motorized, wherein blade 30, 230, 430 may be mechanically connected to a motor (not shown) through elongated member 32, 332, 432 such that blade 30, 230, 430 and/or elongated member 32, 232, 432 may rotate and/or translate. Additionally, the motor may be coupled to a power source. In an exemplary embodiment, the motor may be a small electric screw driver, wherein the power source may be at least one battery. Further, the motor and/or the power source may be situated within or adjacent to elongated member 32. In various embodiments, the motor is coupled to a switch 64 situated along an exterior surface of elongated member 32 for actuating the motor and shaving mechanism 14 (FIG. 7).

With reference to FIG. 3, in various embodiments, shaving mechanism 314 may further include a support plate 34. Support plate 34 may support blade 330 wherein at least a portion of an edge 36 of blade 330 extends beyond support plate 34. Additionally, in various embodiments, support plate 34 may be coupled to blade 330. In various embodiments, blade 330 may include an opening (not shown) such that support plate 34 may be more securely attached to blade 330. Further, in various embodiments, blade 330 may include an opening (not shown) such that elongated member 332 may couple to support plate 34 and blade 330.

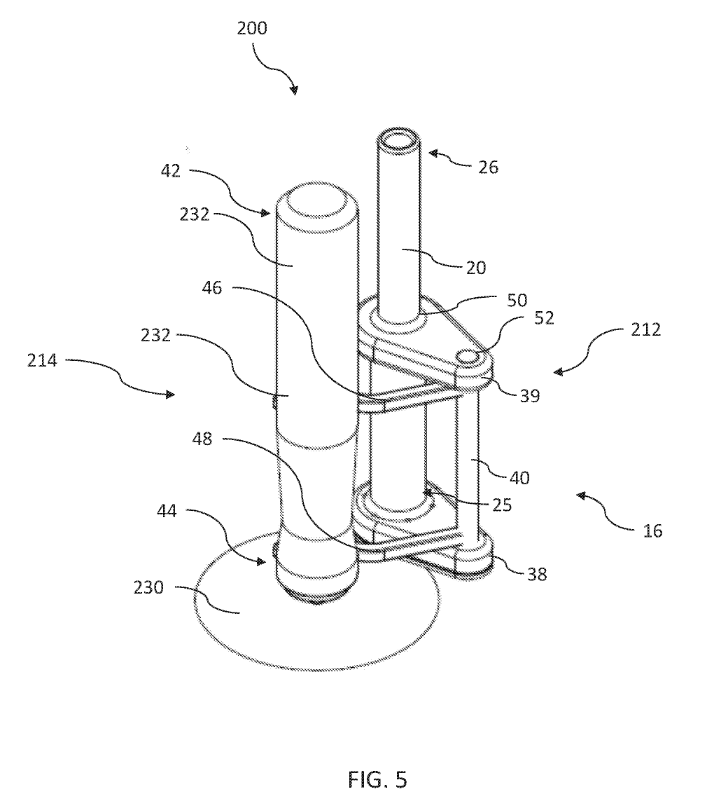

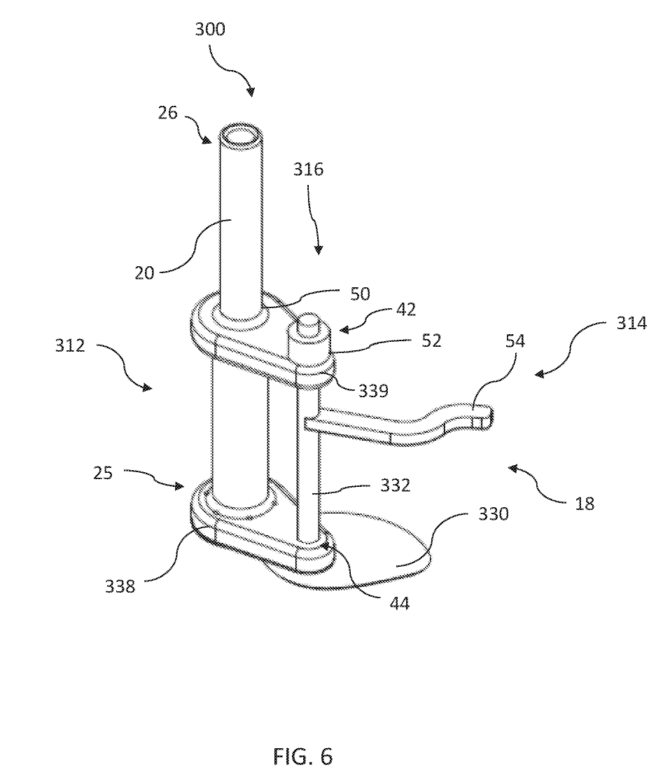

Referring now to FIGS. 5-7, connecting mechanism 16, 316, 416 generally includes at least one link 38, 338, 438 connecting shaving mechanism 214, 314, 414 to suction mechanism 212, 312, 412. Link 38, 338, 438 may connect to elongated member 232, 332, 432 of shaving mechanism 214, 314, 414 and conduit 20 of suction mechanism 212, 312, 412. Furthermore, in various embodiments, connecting mechanism 16, 316, 416 may include two links 38/338/438, 39/339/439, wherein links 38/338/438, 39/339/439 generally each include a first opening 50 and a second opening 52. In various embodiments, link 438 may further include opening 62 (FIG. 7), aligned with opening 58 (FIG. 4), through which arm 60 of the motor of shaving mechanism 414 may extend. Additionally, in various embodiments (as depicted in FIG. 6), first opening 50 of links 338, 339 may receive suction mechanism 312, while second opening 52 may receive shaving mechanism 314. In various embodiments (as depicted in FIGS. 5 and 7), first opening 50 of links 38/438, 39/439 may receive suction mechanism 212/412, while second opening 52 of links 38/438, 39/439 may receive a rod 40. Rod 40 may also include two arms 46/446, 48/448, wherein arms 46/446, 48/448 may be connected to or integral with shaving mechanism 214, 414. In various embodiments, arms 446, 448 may be integral with elongated member 432 of shaving mechanism 414. In addition, in various embodiments, links 38/338/438, 39/339/439 may be integrated with conduit 20 such that conduit 20 and links 38/338/438, 39/339/439 form a single, integral piece. Furthermore, links 38/338/438, 39/339/439 may be positioned such that one link is proximate to distal ends 25, 44 of conduit 20 and elongated member 232/332/432, while the other link is proximate to proximal ends 26, 42 of conduit 20 and elongated member 232/332/432.

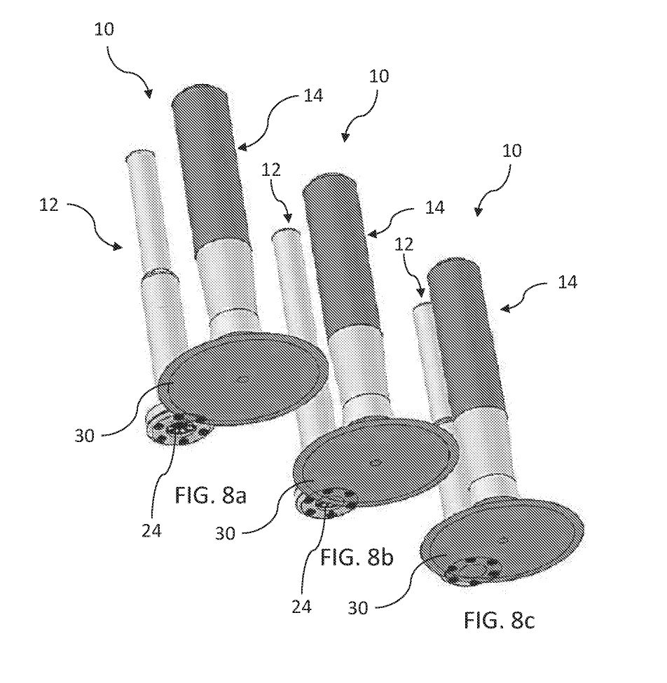

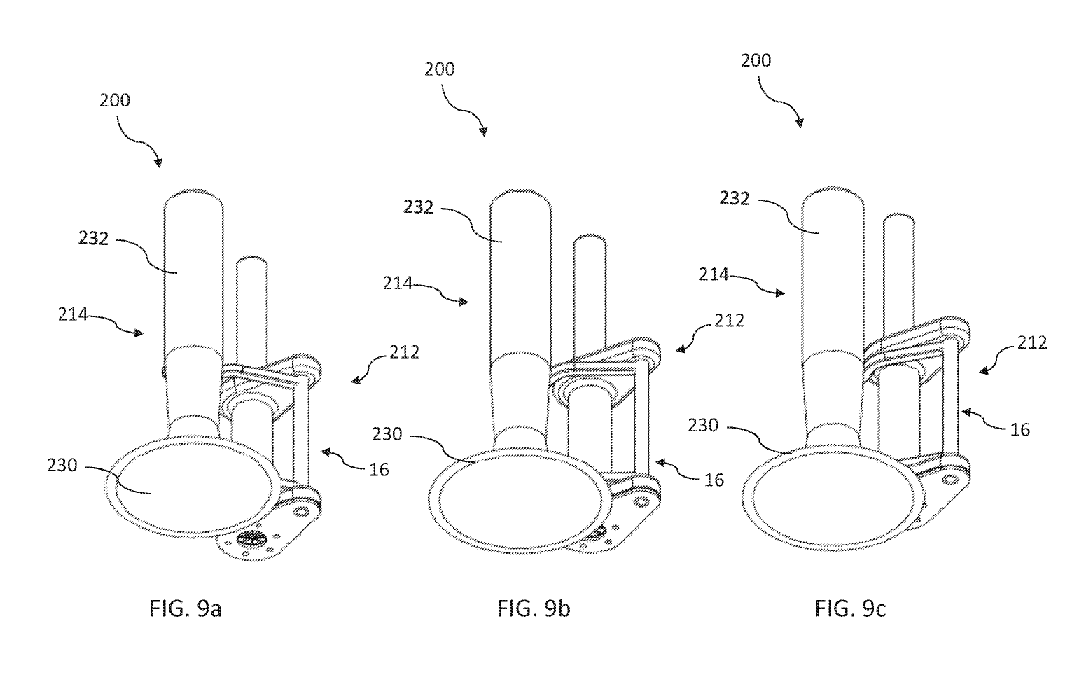

Referring now to FIGS. 8a-c, 9a-c, 10a-c, and 11a-c, biopsy apparatus 10, 200, 300, 400 may be actuated by a user attaching suction mechanism 12, 212, 312, 412 to a suction source and then supporting suction mechanism 12, 212, 312, 412 adjacent to the skin of the patient. Then, rotation of blade 30, 230, 330, 430 may be initiated, if applicable, and the user may manually or mechanically maneuver shaving mechanism 14, 214, 314, 414 across the skin of the patient from an open position (FIGS. 8a, 9a, 10a, and 11a) to a closed position (FIGS. 8c, 9c, 10c, and 11c), wherein blade 30, 230, 330, 430 passes over the skin of the patient until it comes in contact tangentially to the suctioned portion of skin and shaves off the suctioned portion of skin below suction mechanism 12, 212, 312, 412. In various embodiments, the arm of the motor of shaving mechanism 414 may translate within opening 62 when blade 430 translates from the open position to the closed position. Furthermore, the suctioned portion of skin may be retrieved from apparatus 10, 200, 300, 400 by terminating blade 30, 230, 330, 430, if applicable, and the suction source, and transitioning apparatus 10, 200, 300, 400 back to the open position.

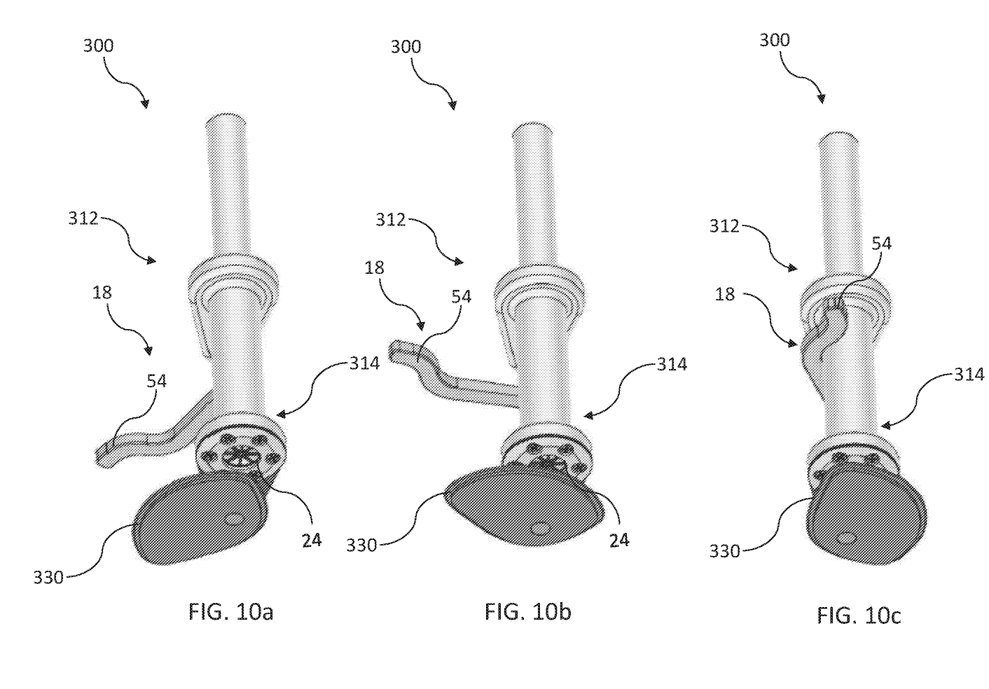

Referring to FIGS. 3, 6, and 10a-c, in various embodiments, biopsy apparatus 300 may also include an actuating mechanism 18. Actuating mechanism 18 may generally include a lever 54 in fixed connection with shaving mechanism 314. To operate a biopsy apparatus with actuating mechanism 18, first, a user may attach suction mechanism 312 to a suction source. Next, with shaving mechanism 314 in an open position (FIG. 10a), suction mechanism 312 may be held in a hand of a user and placed adjacent to the skin desired to be removed. Then, the user may lift suction mechanism 312 up slightly such that shaving mechanism 314 may clear the surrounding skin when making the cut. Finally, the user may actuate lever 54 with the user's other hand or a member of the same hand supporting suction mechanism 312, which allows blade 330 to transition from an open position (FIG. 10a) to a midway position (FIG. 10b) to a closed position (FIG. 10c) below suction mechanism 312. For blade 330 to transition from an open position to a closed position, lever 54 rotates elongated member 332 and blade 330 such that blade 330 passes over the skin of the patient until it comes in contact tangentially to the suctioned portion of skin and shaves off the suctioned portion of skin below suction mechanism 312. The closed position is obtained once edge 36 of blade 330 has eclipsed channel 24. In various embodiments, lever 54 may extend from elongated member 332. To retrieve the shaved skin portion, the user may terminate the suction source, remove the device from the patient and rotate lever 54 to transition blade 330 back to the open position.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

Furthermore, the scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A biopsy apparatus for extracting a quantifiable depth of tissue from skin of a patient comprising:
a suction mechanism comprising a conduit having a proximal end and a distal end, a channel extending from the distal end of the conduit, and a screen in fluid communication with the channel and the conduit, the suction mechanism being coupled to a vacuum source; and
a shaving mechanism comprising a blade.

2. The biopsy apparatus of claim 1, wherein the shaving mechanism further comprises an elongated member coupled to the blade.

3. The biopsy apparatus of claim 1, wherein the screen is positioned between the distal end of the conduit and the channel.

4. The biopsy apparatus of claim 3, wherein the screen is laterally offset from the conduit.

5. The biopsy apparatus of claim 1, further comprising:
a connecting mechanism comprising at least one link coupling the shaving mechanism and the suction mechanism.

6. The biopsy apparatus of claim 1, wherein the channel is created by a perforated plate.

7. The biopsy apparatus of claim 1 further comprising:
an actuating mechanism coupled to the shaving mechanism, whereby the actuating mechanism engages the blade to rotate and move the blade across the skin to remove a portion of skin suctioned by the suction mechanism.

8. The biopsy apparatus of claim 7, wherein the actuating mechanism is one of a lever and a motor.

9. The biopsy apparatus of claim 8, wherein the motor includes a power source and a power switch.

10. The biopsy apparatus of claim 9, wherein the power source is a battery.

11. The biopsy apparatus of claim 9, wherein the power switch is positioned about an exterior surface of the elongated member.

12. The biopsy apparatus of claim 1, wherein the shaving mechanism further includes a support plate coupled to the blade.

13. The biopsy apparatus of claim 1, wherein the blade is round.

14. The biopsy apparatus of claim 1, wherein the screen is formed within a plate.

15. A method for extracting a quantifiable depth of tissue at harvest comprising:
providing a biopsy apparatus, the biopsy apparatus comprising a suction mechanism coupled to a vacuum source and a shaving mechanism, the suction mechanism comprising a conduit, a channel and a screen, wherein the conduit has a proximal end and a distal end, and the screen is in fluid communication with the channel and the conduit, and the shaving mechanism comprising a blade;
positioning the suction mechanism of the biopsy apparatus adjacent to skin of a patient;
actuating the vacuum source, whereby suction is applied to the skin of the patient through the conduit and the channel creating a suctioned portion of skin within the channel; and
actuating the shaving mechanism, whereby the blade passes over the skin tangential to the suctioned portion of skin and under the suction mechanism thereby removing the suctioned portion of skin from the patient.

16. The method of claim 15 further comprising pulling the suction mechanism away from the skin of the patient prior to actuating the shaving mechanism.

17. The method of claim 15, wherein the shaving mechanism is actuated by rotation of a lever coupled to the elongated member.

18. The method of claim 15, wherein the shaving mechanism is actuated by initiating a motor coupled to the elongated member.

19. The method of claim 18, wherein the motor is initiated by toggling a power switch coupled to the elongated member.

20. The method of claim 15, wherein the screen is positioned between the distal end of the conduit and the channel, and a depth of the suctioned portion of skin is determined by the screen.
